(19) United States
(12) Patent Application Publication
(10) Pub. No.: US 2010/0210420 A1
(43) Pub. Date: Aug. 19, 2010

(54) Exercise System and Physiological Information Sensing Device Thereof

(76) Inventors: Tony Chang, Taipei City (TW); Kuang-Chao Fan, Taipei City (TW); Shou-Heng Chen, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/492,210

(22) Filed: Jun. 26, 2009

(30) Foreign Application Priority Data
Feb. 13, 2009 (TW) 098104575

(51) Int. Cl. A63B 71/00; A61B 5/024; A61B 5/01

(52) U.S. Cl. 482/8; 600/508; 600/301

(57) ABSTRACT

An exercise system comprises a main body, at least one force-applied portion, a physiological information sensing device, a processing unit, and a transmission module. The at least one force-applied portion, on which a user can apply force, is connected to the main body. The physiological information sensing device comprises at least one flexible pressure sensing module and a heartbeat sensing unit. The at least one flexible pressure sensing module is connected to the at least one force-applied portion respectively. When the force applied by the user is input to the at least one force-applied portion, the at least one flexible pressure sensing module can produce pressure sensing information. The heartbeat sensing unit can measure heartbeat information. The processing unit is electrically connected to the physiological information sensing device. The processing unit manipulates the pressure sensing information and the heartbeat information and then produces output information. The transmission module is electrically connected to the processing unit and transmits the output information.

### BACKGROUND OF THE INVENTION

1. Field of the Invention

The present invention relates to an exercise system and an information sensing device thereof, and more particularly, to an exercise system comprising a physiological information sensing device and a physiological information sensing device applied to an exercise system.

2. Description of the Related Art

With advances in technology, the quality of life continually improves. With such improvement, people have more time for personal health management. In order to control the state of their health, people regularly keep records of their exercise in addition to having periodic health examinations at hospitals.

According to the American College of Sports Medicine (ACSM), alternating between cardiopulmonary and strength training could be more effective.

However, most general exercise systems separate cardiopulmonary training from strength training, having different exercise equipment for each type. Since it is not convenient to combine the data from the different exercise systems, the users cannot easily control the overall exercise state.

Muscular strength training machines in the prior art usually do not have the function of measuring the heart rate. Moreover, the strength training machines estimate the strength force value of the user by an indirect way. For example, a total force value applied by a user is calculated by an assumed single strength applied by a general user multiplied by frequency. However, the applied force value of each user or each time is not quite the same, so the resulting estimation of force value is not accurate.

Furthermore, in present exercise measurement laboratories, special purpose fixtures are designed to apply load cells to match different constructions of exercise training machines. Because of the higher cost, the exercise training machines are not suitable for commercial usage.

Therefore, it is desirable to provide an exercise system comprising a physiological information sensing device and a physiological information sensing device applied to an exercise system to mitigate and/or obviate the aforementioned problems.

### SUMMARY OF THE INVENTION

A primary object of the present invention is to provide an exercise system with a physiological information sensing device, capable of receiving physiological data during a user's exercise program.

Another object of the present invention is to provide a physiological information sensing device capable of being applied to a force-applied portion of an exercise system.

In order to achieve the above-mentioned objective, the present invention provides an exercise system comprising a main body, at least one force-applied portion, a physiological sensing information device, a processing unit, and a transmission module, wherein the force-applied portion is connected with the main body. The user can apply the force on the at least one force-applied portion; the physiological information sensing device comprises at least one flexible pressure sensing module and a heartbeat sensing unit, and the at least one flexible pressure sensing module is separately connected with the force-applied portion. When the user applies the force on the force-applied portion repeatedly, the flexible pressure sensing module produces pressure information, and the heartbeat sensing unit measures heartbeat information. The processing unit is electrically connected with the physiological information sensing device, and the processing unit processes the pressure information and the heartbeat information to produce output information. The transmission module is electrically connected with the processing unit and can deliver the output information.

In order to achieve another objective, the physiological information sensing device of the present invention comprises a flexible pressure sensing module, a heartbeat sensing unit, and a transmission module. The flexible pressure sensing module can cover the force-applied portion; when the user applies a force on the force-applied portion, the flexible pressure sensing module produces pressure sensing information. The flexible pressure sensing module comprises a plurality of miniature pressure sensing units. The heartbeat sensing unit is connected with the pressure sensing module. When the user touches the heartbeat sensing unit, the heartbeat sensing unit produces heartbeat information. The transmission module is electrically connected with the flexible pressure sensing module and can output the pressure information and heartbeat information.

### BRIEF DESCRIPTION OF THE DRAWINGS

FIG. 1 is a schematic drawing of an embodiment of the exercise system of the present invention.

FIG. 2 is a structural diagram of the exercise system of the present invention.

FIG. 3 is a schematic drawing of the physiological information sensing device of the invention.

FIG. 4 is a schematic drawing of the physiological sensing information system of the invention during operation.

FIG. 5 is a schematic drawing of another embodiment of the exercise system of the present invention.

### DETAILED DESCRIPTION OF THE PREFERRED EMBODIMENT

The advantages and innovative features of the invention will become more apparent from the following detailed description when taken in conjunction with the accompanying drawings.

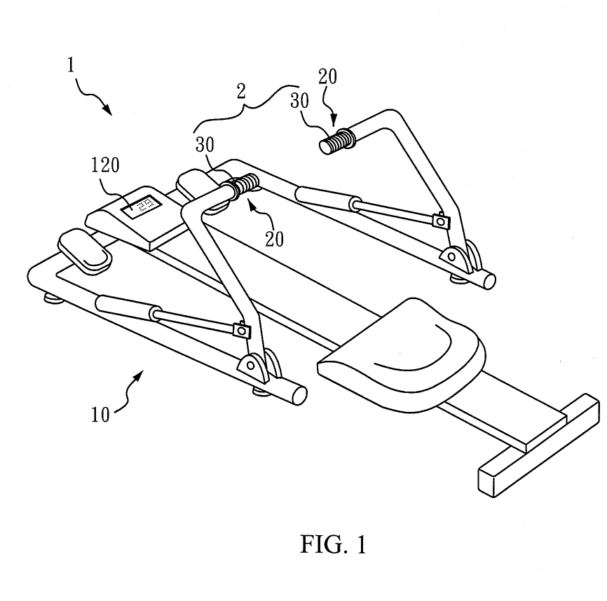

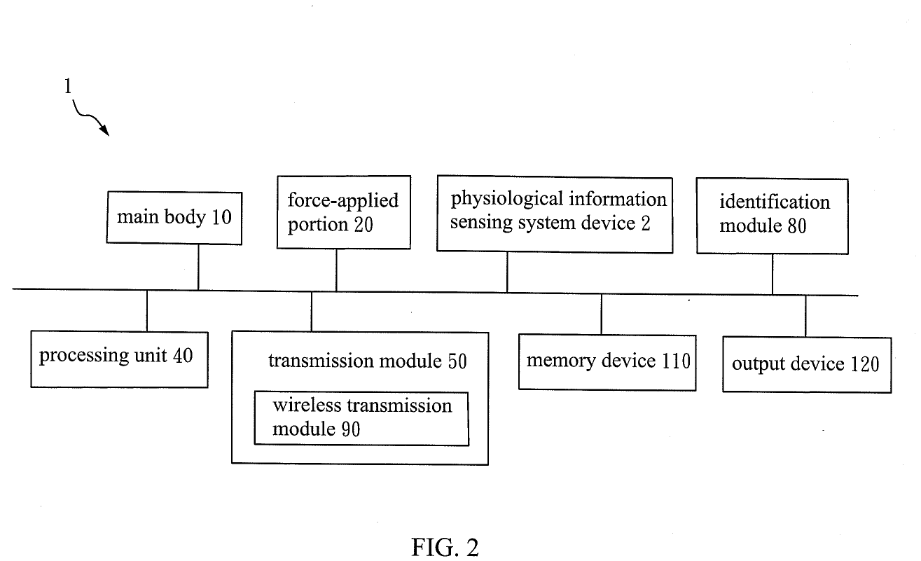

Please refer to FIG. 1 and FIG. 2. FIG. 1 is a schematic drawing of one embodiment of the exercise system of the present invention; FIG. 2 is a structural diagram of the exercise system of the present invention. The exercise system 1 of the present invention comprises a main body 10, at least one force-applied portion 20, a physiological information sensing system device 2, a processing unit 40, a transmission module 50, an identification module 80, a memory device 110, and an output device 120.

The main body 10 is the main mechanical construction of a weight training machine. For example, the main body 10 could be composed of a force-applied portion, a pneumatic/hydraulic cylinder, a linkage rod, a pulley, and/or a loading member. The force-applied portion 20 is connected with the main body 10. In this embodiment, the exercise system 1 is a hand portion weight training machine (as shown in the rowing machine in FIG. 1). Therefore, the force-applied portion 20 is a hand force-applied portion. The number of the force-applied portion 20 is two, so the user's hands operate them separately.

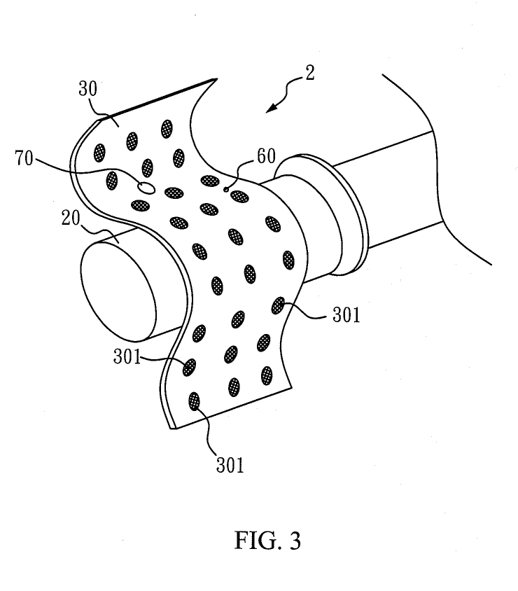

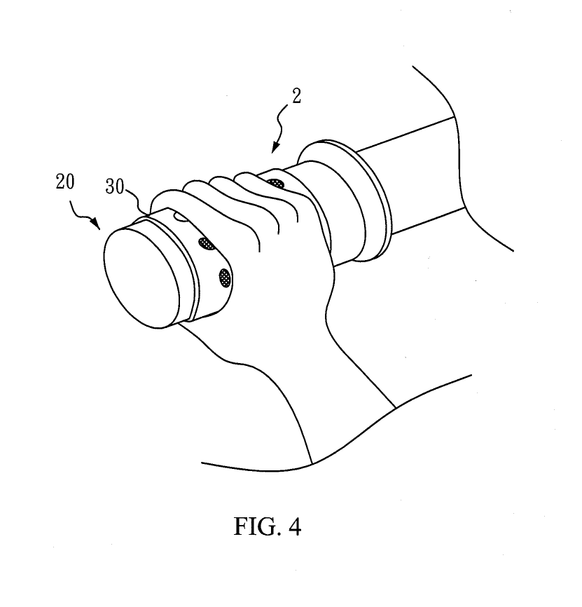

Please refer to FIG. 3 and FIG. 4. FIG. 3 is a schematic drawing of the physiological information sensing device. FIG. 4 is a schematic drawing of the physiological sensing information system during operation. The physiological information sensing device 2 covers a part of the force-applied portion 20 for sensing the exercise state of the user directly. In this embodiment, the physiological information sensing device 2 is separately connected with the force-applied portions 20, 20a of the exercise system 1.

It should be noted that the physiological information sensing device 2 can not only be installed on the exercise system 1 of this invention but can also be applied to other kinds of exercise equipment or devices to add the function of sensing physiological information. For example, the physiological information sensing device 2 can be installed on various kinds of force-applied portions of exercise devices, or on a golf club, a tennis racket, or a badminton racket, etc.

In this embodiment, the physiological information sensing device 2 comprises a flexible pressure sensing module 30, a heart-beat sensing unit 60, and a temperature sensing unit 70. The flexible pressure sensing module 30, the heart-beat sensing unit 60, and the temperature sensing unit 70 can respectively produce pressure information, heartbeat information, and temperature information.

The flexible pressure sensing module 30 can sense pressure. In this embodiment, the flexible pressure sensing module 30 is a sheet, with miniature pressure sensor units 301 separately set on the soft basic board. Depending on the needs of the user, the density of the miniature pressure sensing units 301 can be changed. The sensing principle of the miniature pressure sensor units 301 is via piezoelectricity, resistance, capacitance, or inductance. A miniature pressure sensing unit 301 comprises a piezoelectric sensing unit, a resistance sensing unit, a capacitance sensing unit, and/or an inductance sensing unit. When the status of the miniature pressure sensing units 301 (the structural shape for example) is changed by pressure from the user, the sensing information changes immediately. Since the miniature pressure sensing unit 301 is a kind of prior art, there is no need for further description.

It should be noted that the construction of the flexible pressure sensing module 30 is not limited by the description above. The flexible pressure sensing module 30 measuring the pressure is not restricted to the miniature pressure sensing unit 301. It can be replaced by other types, such as a piezoelectric sensing unit, a resistance sensing unit, a capacitance sensing unit, or an inductance sensing unit. For instance, the flexible pressure sensing module 30 can be a pressure sensor or a strain gauge, a kind of prior art.

In this embodiment, the number of the flexible pressure sensing modules 30 is two, and they are separately connected to two force-applied portions 20. When the user's hands apply force to the force-applied portion 20 (as shown in FIG. 4), the flexible pressure sensing module 30 directly senses the pressure from the hands, wherein the measured information is an unprocessed electrical signal. It should be noted that each sensing unit of the physiological information sensing device 2 (such as the heartbeat sensing unit 60 and temperature sensing unit 70 in this embodiment) can produce sensing information individually.

The processing unit 40 is electrically connected to the physiological information sensing device 2. The processing unit 40 deals with all information (such as pressure, temperature, or pulse information) obtained from the physiological information sensing device 2. By way of the combination of circuits and numerical adjustment, output information is produced. For example, the processing unit 40 can amplify the signal, filter the signal, or analyze information. Therefore, the processing unit 40 deals with and analyzes the “measurement information” sensed by the flexible pressure sensing module 30 to produce the “output information”.

Moreover, when the user repeatedly applies force to the force-applied portion 20, the flexible pressure sensing module 30 produces periodical pressure data. Based on the periodical pressure data produced from the flexible pressure sensing module 30, the processing unit 40 produces the force frequency information.

The transmission module 50 is electrically connected with the processing unit 40. The transmission module 50 can transmit the output information. In this embodiment, the transmission module 50 comprises a wireless transmission module 90 for transmitting the output information produced by the processing unit 40 to an external information gathering device (not shown) via a wireless connection. The external information gathering device can be, for example, a computer device, a cell phone, or a PDA. It should be noted that the transmission module 50 is not limited by the description above. The transmission module 50 can also include a USB transmission module or a local area network (LAN). For example, when the transmission module 50 transmits the information via a wired connection, the transmission module 50 can comprise elements of a wire and a connector.

In this embodiment, the processing unit 40 and the transmission module 50 are set in the main body 10. It should be noted that the positions of the processing unit 40 and the transmission module 50 are not limited by the description above. For example, the processing unit 40 or the transmission module 50 can be set inside the physiological information sensing device 2, or in the force-applied portion 20.

The memory device 110 can temporarily or permanently store different types of measurement information and/or output information for later analysis and application. It should be noted that the memory device 110 can also be an insertable memory device. It should also be noted that the exercise system 1 need not comprise a memory device 110 if the exercise system 1 does not need to store information.

The output device 120 can receive separate kinds of information transmitted from the transmission module 50 and output the measurement information sensed by the physiological information sensing device 2. For example, the output device 120 can be a dot matrix display (as shown in FIG. 1), a seven-segment display, a LCD monitor, a speaker, or a printing device.

The heartbeat measure information is obtained from the heartbeat sensing unit 60. In this embodiment, the heartbeat sensing unit 60 is connected with the flexible pressure sensing module 30 for measuring the heartbeat frequency of the user during the exercise period when the user holds the force-applied portion 20. For example, the heartbeat sensing unit 60 can be an impulse signal sensing unit or a far infrared sensing unit. Furthermore, when the heartbeat frequency is abnormal (too high or too low, for example), the output device 120 will show an image on the display or give another type of warning (a sound or flashing light, for example) to inform the user.

The temperature sensing information is obtained from the temperature sensing unit 70. In this embodiment, the temperature sensing unit 70 is connected to the flexible pressure sensing module 30 for detecting the user's body temperature during the exercise period when the user holds the force-applied portion 20. Furthermore, when the user's body temperature is too high or too low, the output device 120 will show an image in the display or employ another way to inform the user.

It should be noted that the structure of the physiological information sensing device 2 is not limited to the description above. For example, the physiological information sensing device 2 can comprise only the flexible pressure sensing device 30 and the heartbeat sensing unit 60, or the physiological information sensing device 2 can further comprise other sensing units (such as an electrocardiogram sensor, or a blood oxygen saturation sensor) to collect more biomedical data in order to record the exercise condition of the user more accurately. The various types of measurement information can be pressure information, heartbeat information, and temperature information in this embodiment, for example, being transmitted to the processing unit 40 to produce the output information.

The exercise system 1 may be set in an open space such as a gymnasium. Because the exercise system 1 is used by many people, the identification module 80 is used for identifying the users. For example, a users' card can be identified by an infrared ray sensor or a RFID reader, or the user may log in by keying in account and password numbers, allowing the recording of personal information and the setting of long-term personal exercise status information by means of the identification function. For instance, the personal exercising status information can be stored in a memory device carried and kept by the user.

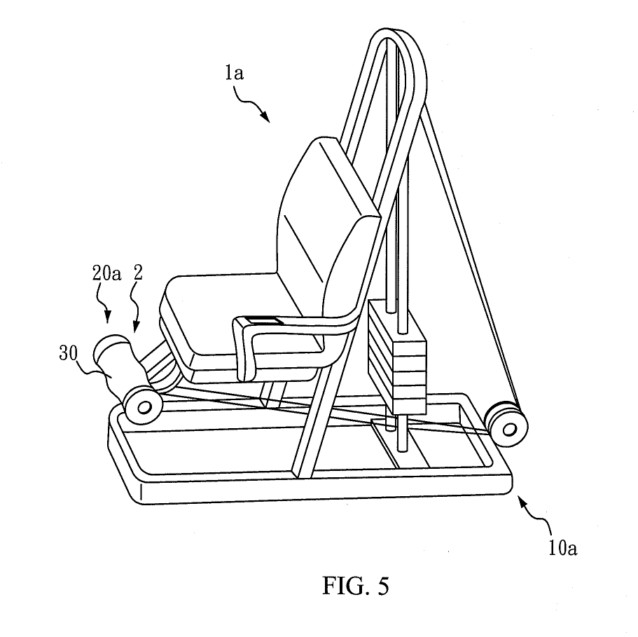

Finally, please refer to FIG. 5. FIG. 5 is a schematic drawing of another embodiment of the exercise system of the present invention. The exercise system 1a comprises a main body 10a, a force-applied portion 20a, a physiological sensing device 2, a processing unit 40, and a transmission module 50. In this embodiment, the exercise system 1a is a leg weight training machine. Therefore, the force-applied portion 20a is a leg force-applied portion, and the number of the force-applied portion 20a is one. Since the constructions of the physiological information sensing device 2, the processing unit 40, and the transmission module 50 are the same as those described previously, no further description is required.

It should be noted that the exercise system 1 and the physiological information sensing device 2 may be applied not only to the weight training machine for hands or legs but also to other weight training machines. For example, the exercise system of the invention can also be applied to machines for training the abdominal muscles or the back muscles, or to a training machine for rehabilitation.

It is noted that the above-mentioned embodiments are only for illustration. It is intended that the present invention cover modifications and variations of this invention provided they fall within the scope of the following claims and their equivalents. Therefore, it will be apparent to those skilled in the art that various modifications and variations can be made to the structure of the present invention without departing from the scope or spirit of the invention.

What is claimed is:

1. An exercise system, used for a user to apply a force, the exercise system comprising:
a main body;
at least one force-applied portion connected with the main body;
the user can apply the force on the at least one force-applied portion;
a physiological information sensing device comprising:
at least one flexible pressure sensing module connected with the at least one force-applied portion separately; when the user applies the force on the at least one force-applied portion, the at least one flexible pressure sensing module produces pressure information; and
a heartbeat sensing unit, the heartbeat sensing unit measuring heartbeat information;
a processing unit electrically connected with the physiological information sensing device; the processing unit processing the pressure information and the heartbeat information to produce output information; and
a transmission module electrically connected with the processing unit; the transmission module can deliver the output information.

2. The exercise system as claimed in claim 1, wherein the flexible pressure sensing module comprises a plurality of miniature pressure sensing units.

3. The exercise system as claimed in claim 2, wherein each miniature pressure sensing unit comprises:
a piezoelectric sensing unit, a resistance sensing unit, a capacitance sensing unit, or an inductance sensing unit.

4. The exercise system as claimed in claim 1, wherein the number of the at least one flexible pressure sensing module is two; the number of the at least one force-applied portion is two.

5. The exercise system as claimed in claim 1, wherein the at least one flexible pressure sensing module separately covers a part of the at least one force-applied portion.

6. The exercise system as claimed in claim 1, wherein the heartbeat sensing unit is an impulse signal sensing unit or a far infrared sensing unit.

7. The exercise system as claimed in claim 1, wherein the exercise system further comprises an identification module electrically connected with the processing unit.

8. The exercise system as claimed in claim 1, wherein the physiological information sensing device further comprises a body temperature sensing unit capable of sensing temperature information.

9. The exercise system as claimed in claim 8, wherein the heartbeat sensing unit connects with the flexible pressure sensing module, and the temperature sensing unit connects with the flexible pressure sensing module.

10. The exercise system as claimed in claim 8, wherein the exercise system further comprises an identification module electrically connected with the processing unit.

11. The exercise system as claimed in claim 1, wherein the exercise system is a weight training system, and each force-applied portion is a hand force-applied portion or a leg force-applied portion.

12. The exercise system as claimed in claim 11, wherein repeated application of force on the force-applied portion by the user causes the processing unit to produce applied frequency information.

13. The exercise system as claimed in claim 1, wherein the transmission module comprises a wireless transmission module for outputting the output information to an external information-collecting device.

14. The exercise system as claimed in claim 1, wherein the exercise system further comprises a memory device electrically connected with the processing unit and the transmission module, the memory device being used for storing the pressure information, the heartbeat information, or the output information.

15. The exercise system as claimed in claim 1, wherein the exercise system further comprises an output device electrically connected with the transmission module, the output device being able to display the output information.

16. The exercise system as claimed in claim 10, wherein the exercise system further comprises:
a memory device electrically connected with the processing unit and the transmission module, the memory device being used for storing the pressure information, the heartbeat information and the output information; and
an output device electrically connected with the transmission module, the output device being able to display the output information.

17. A physiological information sensing device, connected with a force-applied portion of an exercise system on which a user can apply a force, the physiological information sensing device comprising:
a flexible pressure sensing module, which can cover the force-applied portion; when the user applies force on the force-applied portion, the flexible pressure sensing module produces pressure sensing information, the flexible pressure sensing module comprising a plurality of miniature pressure sensing units;
a heartbeat sensing unit connected with the flexible pressure sensing module; when the user touches the heartbeat sensing unit, the heartbeat sensing unit produces heartbeat information; and
a transmission module electrically connected with the flexible pressure sensing module; the transmission module can output the pressure information and the heartbeat information.

18. The physiological information sensing device as claimed in claim 17, wherein the physiological information sensing device further comprises:
a body temperature sensing unit connected with the flexible pressure sensing module; when the user touches the body temperature sensing unit, the body temperature sensing unit produces body temperature information.

19. The physiological information sensing device as claimed in claim 17, wherein the flexible pressure sensing module comprises a piezoelectric sensing unit, a resistance sensing unit, a capacitance sensing unit, or an inductance sensing unit; the heartbeat sensing unit is an impulse signal sensing unit or a far infrared sensing unit.

20. The physiological information sensing device as claimed in claim 17, wherein the physiological information sensing device further comprises:
a processing unit connected with the flexible pressure sensing module; the processing unit is electrically connected with the plurality of miniature pressure sensing units and the heartbeat sensing unit; the processing unit processes the pressure information and the heartbeat information to produce output information.
